(19) United States
(12) Patent Application Publication
(10) Pub. No.: US 2018/0207120 A1
(43) Pub. Date: Jul. 26, 2018

(54) COMPOSITIONS AND METHODS FOR TREATING, PREVENTING, OR ALLEVIATING OBESITY OR RELATED DISEASES

(71) Applicant: Korea University Research and Business Foundation, Seoul (KR)

(72) Inventors: Sung-Joon Lee, Seoul (KR); Chunyan Wu, Seoul (KR)

(73) Assignee: Korea University Research and Business Foundation

(21) Appl. No.: 15/861,239

(22) Filed: Jan. 3, 2018

Related U.S. Application Data
(63) Continuation of application No. PCT/KR2016/006859, filed on Jun. 27, 2016.

(30) Foreign Application Priority Data
Jul. 3, 2015 (KR) 10-2015-0095529

(51) Int. Cl. A61K 31/20; A61P 3/04

(52) U.S. Cl. CPC A61K 31/20; A61P 3/04

(57) ABSTRACT

A composition comprising azelaic acid as an active ingredient suppresses accumulation of lipids in adipose tissue and improves lipid metabolism in adipose tissue.

### CROSS-REFERENCE TO RELATED APPLICATIONS

This application is a continuation application of International Application No. PCT/KR2016/006859 filed on Jun. 27, 2016, which claims priority to Korean Application No. 10-2015-0095529 filed one Jul. 3, 2015, which applications are incorporated herein by reference.

### TECHNICAL FIELD

The present invention relates to a pharmaceutical composition or a functional food composition comprising azelaic acid as an active ingredient. More particularly, it relates to a pharmaceutical composition or a health functional food composition comprising azelaic acid, as an active ingredient, which has effects of increasing triglyceride hydrolysis in adipose tissue and suppressing accumulation of triglycerides through the cAMP-PKA-HSL signaling pathway by binding to and subsequent activation of olfactory receptor 544 (Olfr544), a G-protein coupled receptor (GPCR) expressed in the cell membrane of adipose tissue.

### BACKGROUND ART

Excessive accumulation of energy in the body causes various metabolic diseases. Obesity, which is a representative disease caused by impairment of the balance of body energy metabolism, leads to insulin resistance and results in other diseases, such as hyperlipidemia, hypertension and the like. It is well-known that it is essential to regulate a life-style to suitably maintain body energy metabolism, and obesity may be treated to some extent by increasing an exercise amount and decreasing food intake through proper diet.

Although change in diet and a life-style, including exercise, is most important, treating obesity only by modifying the lifestyle is difficult for most chronic disease patients. Instead, healthcare management including drug therapy using a lipid lowering agent is required. Several drugs for treating obesity have been developed and used. However, agents suppressing appetite by acting on the central nervous system, for example, fenfluramine/phentermine or sibutramine, have problems of severe side effects, including development of cardiovascular diseases and the like, such that commercialized drugs were withdrawn from the market. Orlistat, an oral lipase inhibitor, is not effective for some ethnic groups, who do not eat high-fat diets. Therefore, existing drugs are not sufficiently effective and thus there is a need to develop a novel agent. Meanwhile, as many people are reluctant and burdened to take drugs, a need for research on active ingredients capable of treating or preventing obesity or chronic diseases using a safe food ingredient or an agent naturally produced in the body has increased.

The chemical name of azelaic acid is nonanedioic acid, a dicarboxylic acid with 9 carbon atoms (FIG. 1). Azelaic acid is produced during the omega-oxidation process or as a peroxide of linoleic acid in the body. Alternatively, azelaic acid is also naturally produced in various grains, such as wheat, barley, oatmeal, sorghum and the like, thereby having been ingested in a form of food. Studies hitherto showed that azelaic acid is effective to treat inflammatory skin diseases, such as flushing, acne and the like. Additionally, effects of azelaic acid on arteriosclerosis, blood glucose control, anti-cancer and the like have been reported as well. Nevertheless, neither studies on azelaic acid in connection with the effect of reducing body fat nor studies on the mechanism underlying this effect have been reported. Furthermore, studies on the detailed mechanism, such as identification of a target protein and the like, have not been made.

Olfr544 is an olfactory receptor (hereinafter, referred as ‘Olfr544’). Generally, olfactory receptors are expressed in olfactory epithelial cells and transmit olfactory information to the cerebrum. However, recent reports showed that olfactory receptors are expressed in various tissues in addition to olfactory tissues. Humans have 400 or more olfactory receptor genes, which correspond to 1-2% of the human genome. Hence, it is not surprising that olfactory receptors that occupy such a large portion of the genome perform necessary functions in general tissue cells in addition to transmitting olfactory information.

Recent studies reported various functions of olfactory receptors in general tissues. For instance, studies showed that an olfactory receptor has a function of detecting a pheromone material in the sperm, OR2AT4 plays a role in skin regeneration in the keratinocytes of the skin, and Olfr78 is expressed in kidney tissue and regulates secretion of renin hormone and the like. In such a way, novel functions of olfactory receptors have been reported by many researchers, including the present inventors who published that OR1A1 is expressed in liver tissue and regulates triglyceride metabolism in liver tissue (Wu C et al., Int. J. Biochem. Cell Biol., 64:75-80, 2015).

The present inventors aimed to develop a pharmaceutical composition or a functional food composition that contains azelaic acid capable of improving lipid metabolism (alleviating obesity) by effectively reducing triglyceride levels in adipose tissue and thus decreasing body fat. The present inventors disclose that azelaic acid suppresses lipid accumulation in adipose tissue by functioning as a ligand for Olfr544, an olfactory receptor ectopically expressed in 3T3-L1 adipocytes, to activate the cAMP-PKA-HSL signaling pathway.

### SUMMARY

One of the objects of the present invention is to provide a composition for improving lipid metabolism in adipose tissue (composition for alleviating obesity), which composition comprises azelaic acid having effects of increasing triglyceride hydrolysis in adipose tissue and effectively suppressing accumulation of body fat.

In one aspect, the present invention provides a pharmaceutical composition comprising azelaic acid as an active ingredient for preventing or treating obesity.

In another aspect, the present invention provides a health functional food composition comprising azelaic acid as an active ingredient for alleviating obesity.

In still another aspect, the present invention provides a method of preventing, treating, or alleviating obesity by administering azelaic acid.

### BRIEF DESCRIPTION OF DRAWINGS

FIG. 1. shows the chemical structure, the molecular formula, and the molecular weight of azelaic acid (trans azelaic acid).

FIGS. 2A to 2D show that Olfr544, a target molecule of azelaic acid, is significantly expressed in 3T3-L1 adipocytes and mouse adipose tissue. Expression of Olfr544 is analyzed by a reverse transcription-polymerase chain reaction (RT-PCR) experiment (FIG. 2A), a quantitative real-time polymerase chain reaction (qPCR) experiment (FIG. 2B), a western blotting experiment (FIG. 2C), and an immunohistochemistry experiment (FIG. 2D).

FIG. 3 is a graph representing the result of the cytotoxicity test of azelaic acid in 3T3-L1 adipocytes, as measured by the MTT assay.

FIG. 4 shows that Olfr544 activation by azelaic acid stimulates cAMP response element-binding protein (CREB) activity. It represents the result of the reporter gene assay following introduction of an Olfr544 expression vector and a CRE-Luciferase reporter vector into a Hana3A cultured cell line in which expression efficiency of an olfactory receptor is increased to assess the effect (activity) of azelaic acid as an Olfr544 ligand. An EC₅₀ value means the concentration of an agonist at 50% of the maximum efficiency.

FIGS. 5A to 5C show that Olfr544 activation by azelaic acid stimulates cAMP levels in 3T3-L1 adipocytes. FIGS. 5A to 5C represent the concentrations of main secondary messengers involved in intracellular signal transduction pathways, i.e., cAMP (FIG. 5A), IP-one: a metabolite of IP₃ (FIG. 5B), and intracellular calcium (FIG. 5C), after treating 3T3-L1 adipocytes with azelaic acid (AzA). AzA, azelaic acid; A-23187, 10 μM, a calcium-ionophore positive control.

FIG. 6 shows that Olfr544 activation by azelaic acid stimulates the PKA activity in 3T3-L1 adipocytes. It represents the protein kinase A (PKA) activity by treating 3T3-L1 adipocytes with azelaic acid (AzA) for 30 minutes. FSK, forskolin 1 μM; AzA, azelaic acid 50 μM. One-way ANOVA followed by a Tukey's HSD test was used for multiple-group comparisons. Different alphabet letters indicate a significant difference at P<0.05 for multiple-group comparisons.

FIGS. 7A and 7B show that Olfr544 activation by azelaic acid stimulates hormone-sensitive lipase phosphorylation levels in 3T3-L1 adipocytes. FIGS. 7A and 7B represent phosphorylation (phosphorylation level) of cAMP-response element binding protein (CREB) and hormone-sensitive lipase (HSL), target proteins of PKA, by treating 3T3-L1 adipocytes with azelaic acid (AzA) for 2 hours. FIG. 7A is a Western blotting result, and FIG. 7B is a graph showing quantified levels of phosphorylated HSL, total HSL and phosphorylation of HSL relative to HSL protein expression. C (control), negative control; 1 μM FSK (forskolin), positive control; 50 μM AzA (Azelaic Acid).

FIGS. 8A to 8C shows that Olfr544 activation by azelaic acid stimulates lipolysis in 3T3-L1 adipocytes. FIGS. 8A to 8C represent the concentrations of intracellular lipids and amounts of glycerol released by triglyceride hydrolysis by treating 3T3-L1 adipocytes with azelaic acid (AzA) for 2 hours. FIG. 8A represents the concentrations of intracellular triglyceride. FIG. 8B represents the fold changes of the concentrations of intracellular cholesterol. FIG. 8C represents the amounts of glycerol released. C, negative control; FSK, forskolin 1 μM; AzA, azelaic acid 50 μM. One-way ANOVA followed by a Tukey's HSD test was used for multiple-group comparison. Different letters indicate a significant difference at P<0.05 for multiple-group comparison.

FIGS. 9A and 9B show inhibited expression of Olfr544 by transfecting 3T3-L1 adipocytes with an Olf544 shRNA, as analyzed by a RT-PCR experiment (FIG. 9A) and a western blotting experiment (FIG. 9B). FIG. 9C is a graph showing quantified relative protein expression levels. Scr, scrambled shRNA.

FIGS. 10A to 10D show that the effect of azelaic acid on PKA-HSL signaling is dependent on Olfr544 in 3T3-L1 adipocytes. FIGS. 10A to 10C represent the concentrations of intracellular cAMP (FIG. 10A), the relative PKA activity (FIG. 10B), and phosphorylation (phosphorylation levels) of HSL (FIG. 10C) by treating 3T3-L1 adipocytes in which the Olfr544 expression was knocked-down by Olf544 shRNA with azelaic acid (AzA) for 2 hours. FIG. 10D is a graph showing quantified relative protein expression levels. Scr, scrambled shRNA; C, control; FSK, forskolin 1 μM; AzA, azelaic acid 50 μM.

FIG. 11 shows that the effect of azelaic acid on lipolysis is dependent on Olfr544 in 3T3-L1 adipocytes. It represents the amounts of glycerol released by lipolysis by treating 3T3-L1 adipocytes in which the Olfr544 expression was knocked-down by an Olf544 shRNA with azelaic acid (AzA) for 2 hours. Scr, scrambled shRNA; C, control; FSK, forskolin 1 μM; AzA, azelaic acid 50 μM.

FIGS. 12A and 12B show that azelaic acid administration reduces body weight in HFD-fed ob/ob mice. FIGS. 12A and 12B represent the changes in body weight (FIG. 12A) and feed intake amount (FIG. 12B) while orally administering azelaic acid (AzA) into genetically obesity-induced ob/ob mice at a concentration of 50 mg/kg for 6 weeks.

FIG. 13 shows that azelaic acid administration reduces adiposity in HFD-fed ob/ob mice. The graph shows the levels of total fat (Total), abdominal fat (Adb), and subcutaneous fat (SubQ) measured by the micro-CT method after orally administering azelaic acid (AzA) into genetically obesity-induced ob/ob mice at a concentration of 50 mg/kg for 6 weeks.

FIGS. 14A to 14C show that azelaic acid administration reduces cholesterol levels in HFD-fed ob/ob mice. FIGS. 14A to 14C represent the concentrations of triglyceride (FIG. 14A), adiponectin (FIG. 14B), and cholesterol (FIG. 14C) in the blood after orally administering azelaic acid (AzA) into genetically obesity-induced ob/ob mice at a concentration of 50 mg/kg for 6 weeks.

FIGS. 15A to 15C show liver weights and liver enzyme levels in HFD-fed ob/ob mice. FIGS. 15A to 15C represent the liver weights (FIG. 15A) and the levels of alanine aminotransferase (ALT) (FIG. 15B) and aspartate aminotransferase (AST) (FIG. 15C), which are hepatotoxicity indeces, after orally administering azelaic acid (AzA) into genetically obesity-induced ob/ob mice at a concentration of 50 mg/kg for 6 weeks.

FIGS. 16A to 16E show that azelaic acid administration reduces respiratory quotient and induces fatty acid oxidation, and shifts fuel preference to fats in HFD-fed WT mice. FIG. 16 represents the total energy expenditure (EE) (FIG. 16A), the respiratory quotient (RQ) (FIG. 16B), fatty acid oxidation (FIG. 16C), the consumption amount of O₂ (FIG. 16D), and the production amount of CO₂ (FIG. 16E) using the indirect calorimetry after orally administering azelaic acid (AzA) into normal mice at a concentration of 50 mg/kg for 6 weeks.

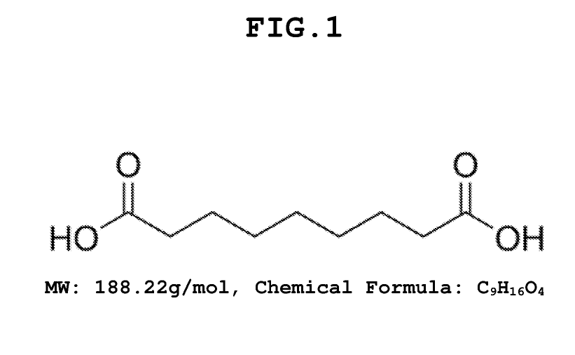

### DETAILED DESCRIPTION

Unless defined otherwise herein, all technical and scientific terms used in the present specification have the meanings commonly understood by a person ordinarily skilled in the art to which the present invention pertains. Generally, nomenclature used in the present specification is well known and commonly used in the art.

In the present invention, upon treatment of 3T3-L1 adipocytes with azelaic acid, azelaic acid has an effect of increasing triglyceride hydrolysis and suppressing triglyceride accumulation by activating olfactory receptor, Olfr544. More specifically, to evaluate the suppressing effect of azelaic acid on adipose triglyceride accumulation, the triglyceride levels in the 3T3-L1 adipocytes treated with azelaic acid, and the amounts of glycerol released as the product of triglyceride hydrolysis were analyzed, thereby evaluating the effect on triglyceride hydrolysis in 3T3-L1 adipocytes. Further, in the present invention, a mechanism underlying the effect of azelaic acid on triglyceride hydrolysis was identified to be mediated via the c-AMP-PKA-HSL signaling pathway through Olfr544 by analyzing characteristics of azelaic acid as an Olfr544 agonist in 3T3-L1 adipocytes and a Hana3A cell line in which Olfr544 is expressed.

In the present invention, upon oral administration of azelaic acid, which is an Olfr544 agonist, to genetically obesity-induced ob/ob mice for 6 weeks, body fat was decreased due to an increase of lipid oxidation, as analyzed by a bio-metabolic rate experiment.

Azelaic acid is naturally produced in the human body or is contained, as a food ingredient, in grains, such as barley, oats, rye and the like, which people eat as main food. According to previous reports, the concentration of azelaic acid in a human body is about 10 to 50 μM, and it is widely known that generally, azelaic acid can be safely ingested. Further, in the MTT toxicity test according to the present invention, toxicity was not exhibited when 3T3-L1 adipocytes were treated with azelaic acid at a concentration of up to 500 μM.

Accordingly, one aspect of the present invention provides a pharmaceutical composition comprising azelaic acid as an active ingredient for treating obesity.

In another aspect, the present invention provides a method of preventing, treating, or alleviating obesity by administering azelaic acid to a subject.

In still another aspect, the present invention provides uses of azelaic acid for medicament to prevent, treat, or alleviate obesity.

In the present invention, treatment of obesity may be characterized by a decrease in triglyceride in adipose tissue, suppression of lipid accumulation in adipose tissue, a decrease in body weight, or a decrease in body fat. Azelaic acid may increase the activity of an olfactory receptor (OR), wherein the olfactory receptor may be olfactory receptor 544 (Olfr544). Further, azelaic acid may increase triglyceride hydrolysis in adipose tissue.

It is known that accumulation of fat (or lipid) and excessive energy in adipose cells may cause obesity and various lipid-associated metabolic diseases, such as diabetes mellitus, hyperlipidemia, fatty liver, arteriosclerosis, hypertension and cardiovascular diseases, or metabolic syndromes in which the above-mentioned diseases are simultaneously and multiply developed, and the like. The metabolic syndrome is a syndrome in which risk factors, such as hyperlipidemia, hypertension, glucose metabolism disorder and obesity, simultaneously appear. Recently, this syndrome was officially named as the metabolic syndrome or the insulin resistance syndrome by Adult Treatment Program III (ATP III) established by the World Health Organization and National Heart, Lung, and Blood Institute (NHLBI) of the National Institute of Health (NIH).

The term “fatty liver” as used herein means a state in which fat is excessively accumulated in liver cells due to the fat metabolism disorder in the liver and causes various diseases, such as angina, myocardial infarction, stroke, arteriosclerosis, fatty liver, pancreatitis and the like.

In accordance with embodiments of the present invention, azelaic acid obtained by the juice extraction, the hot water extraction, the ultrasonic extraction, the solvent extraction, or the reflux cooling extraction methods may be used, wherein the solvent extraction may use ethanol, methanol, acetone, hexane, ethyl acetate, methylene chloride, water, or a combination thereof. Azelaic acid may be extracted by various methods generally used to prepare natural azelaic acid.

The term “azelaic acid” as used herein may be used interchangeably with a synonym, nonanedioic acid.

Since azelaic acid according to the present invention is a natural material, azelaic acid does not have toxicity at all, such that a large amount of azelaic acid may be continuously used as a drug.

The composition comprising azelaic acid according to the present invention may be formulated with or used in combination with other drugs already used, such as anti-histamine agents, analgesic agents, anti-cancer agents, antibiotic agents and the like.

Unless otherwise described, the term “treat” as used herein means that one or more symptoms of the disease to which the term is applied is reversed or alleviated, or progress of the disease is suppressed or prevented. As used herein, the term “treatment” means a treating behavior when the term “treat” is defined as described above.

The term “pharmaceutical composition” or “medicinal composition” means a mixture of a compound comprising azelaic acid according to the present invention and other chemical ingredients, such as diluents or carriers.

The term “physiologically acceptable” is defined as carriers or diluents that do not damage biological activities and physical properties of the compound.

The term “carrier” or “vehicle” is defined as compounds that allow a target compound to be easily introduced into cells or tissue. For example, dimethylsulfoxide (DMSO) is a carrier generally used to allow various organic compounds to be easily introduced into cells or tissue of living organisms.

The term “diluent” is defined as compounds that stabilize a biologically active form of a target compound and are diluted by water dissolving the compound. A salt dissolved in a buffer solution is used as a diluent in the art to which the present invention pertains. A generally used buffer solution is phosphate buffered saline, as it imitates a salt form of the human body fluid. Since a buffer solution can control a pH of a solution at a low concentration, it is rare for a buffer diluent to alter the biological activity of a compound.

Azelaic acid may be administered to human patients. It may be administered as a pharmaceutical composition in which zelaic acid is combined with other active ingredients or suitable carriers or excipients, as in the combination therapy.

A medicinal composition or a pharmaceutical composition suitable to be used in the present invention includes a composition in which active ingredients are contained in effective amounts enough to achieve desired objects. More specifically, a therapeutically effective amount means an amount of a compound effective for extending survival of an object to be treated, or preventing, reducing or alleviating symptoms of diseases. Particularly, in view of detailed description of the disclosure provided herein, the therapeutically effective amount may be determined by those skilled in the art.

A therapeutically effective amount of the compound comprising azelaic acid according to the present invention may be initially determined using cell culture analysis. For example, a dose may be calculated using an animal model to obtain a circulating concentration range including the half maximal inhibitory concentration (IC₅₀ or the half maximal effective concentration (EC₅₀) determined using cell culture. This information may be used to more accurately determine an effective dose for human.

An administration dose of the azelaic acid may be changed within the above-mentioned range depending on applied administration formulations and intended administration routes. Accurate calculation, administration routes, and administration doses may be selected by individual doctors in consideration of states of patients (for example, see Fingl et al., 1975, “The Pharmacological Basis of Therapeutics”, Ch. 1, p. 1).

A preferable administration dose of azelaic acid according to the present invention may be changed depending on a condition and weight of a patient, a state of disease, a drug formulation, and an administration route and duration, but appropriately selected by those skilled in the art. Generally, a dose range of the composition administrated to a patient may be about 0.5 to 1000 mg/kg based on a body weight of a patient. However, to obtain a preferable effect, azelaic acid according to the present invention may be administered at a daily dose of 0.0001 to 200 mg/kg, preferably 0.001 to 100 mg/kg. One dose may be administered once a day, or divided into several doses and administered. Accordingly, the scope of the present invention is not limited by the administration dose.

Azelaic acid according to the present invention may be administered to mammals, such as rats, mice, livestock, humans and the like, through various routes. All administration methods may be contemplated. For example, azelaic acid may be orally administered, or administered by rectal injection, intravenous injection, intramuscular injection, subcutaneous injection, intrauterine dural injection or intracerebroventricular injection.

In embodiments of the present invention wherein the composition according to the present invention is provided as a mixture containing additional ingredients in addition to azelaic acid, the composition may contain azelaic acid in an amount of 0.001 wt % to 99.9 wt %, preferably, 0.1 wt % to 99 wt %, and more preferably, 1 wt % to 50 wt % based on the total weight of the composition.

A pharmaceutical administration form of the composition according to the present invention may be a form of a pharmaceutically acceptable salt thereof. Further, the composition may be used alone, or as a suitable mixture of, or in combination with other pharmaceutically active compounds.

The term “pharmaceutically acceptable salt” means a form of a compound that does not cause severe stimulation on an organism to which the compound is administered and does not damage biological activities and physical properties of the compound. The terms “hydrate”, “solvate”, and “isomer” also have the same meaning as described above. The pharmaceutically acceptable salts may be obtained by reacting the compound comprising azelaic acid according to the present invention with an inorganic acid, such as hydrochloric acid, bromic acid, sulfuric acid, nitric acid, phosphoric acid and the like; a sulfonic acid, such as methanesulfonic acid, ethanesulfonic acid, p-toluenesulfonic acid and the like; or an organic carbonic acid, such as tartaric acid, formic acid, citric acid, acetic acid, trichloroacetic acid, trifluoroacetic acid, capric acid, isobutanoic acid, malonic acid, succinic acid, phthalic acid, gluconic acid, benzoic acid, lactic acid, fumaric acid, maleic acid, salicylic acid and the like. Additionally, the pharmaceutically acceptable salts may be obtained by reacting the compound containing azelaic acid according to the present invention with a base to form an alkali metal salt, such as an ammonium salt, a sodium salt, a potassium salt and the like; an alkali earth metal salt, such as a calcium salt, a magnesium salt and the like; a salt of an organic base, such as dicyclohexylamine, N-methyl-D-glucamine, tris(hydroxymethyl)methylamine and the like; or a salt of amino acid, such as arginine, lysine and the like.

The pharmaceutical composition or medicinal composition comprising azelaic acid according to the present invention may be formulated in a form for oral administration, such as powders, granules, tablets, capsules, suspensions, emulsions, syrups, aerosols and the like; for external application, suppository, and sterile injection solutions according to methods generally used in the art, respectively. Examples of carriers, excipients and diluents acceptable for use in the composition comprising azelaic acid may include lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinyl pyrrolidone, water, methylhydroxybenzoate, propylhydroxybenzoate, talc, magnesium stearate, and mineral oil.

In embodiments of the present invention wherein the pharmaceutical composition is formulated, diluents or excipients generally used in the art, such as fillers, extenders, binders, wetting agents, disintegrants, surfactants and the like, may be used. Solid formulations for oral administration include tablets, pills, powders, granules, capsules and the like, and may be prepared by mixing azelaic acid with at least one or more excipients, for example, starch, calcium carbonate, sucrose or lactose, gelatin and the like. Additionally, lubricants, such as magnesium stearate or talc, may be used in addition to simple excipients. Liquid formulations for oral administration include suspensions, solutions, emulsions, syrups and the like, and may contain various excipients, such as wetting agents, sweeteners, flavoring agents, preservatives and the like, in addition to water and liquid paraffin, which are generally used as simple diluents. Formulations for parenteral administration include sterile aqueous solutions, non-aqueous solvents, suspensions, emulsions, freeze-dried formulations, and suppositories. In non-aqueous solvents or suspensions, propylene glycol, polyethylene glycol, vegetable oil, such as olive oil, injectable esters, such as ethyloleate, and the like, may be used. As a base for suppositories, witepsol, macrogol, tween 60, cacao butter, laurinum, glycerol-gelatin and the like may be used.

Olfactory receptor is the largest protein group among the G-protein coupled receptor (GPCR) protein superfamily, and generally the expression thereof on the cell membrane is not high. Therefore, expression (the expression level) of olfactory receptor Olfr544 in adipose tissue (or 3T3-L1 adipocytes), which was previously identified in the microarray study by the present inventors, was analyzed through RT-PCR, qPCR, western blotting and immunohistochemistry experiments.

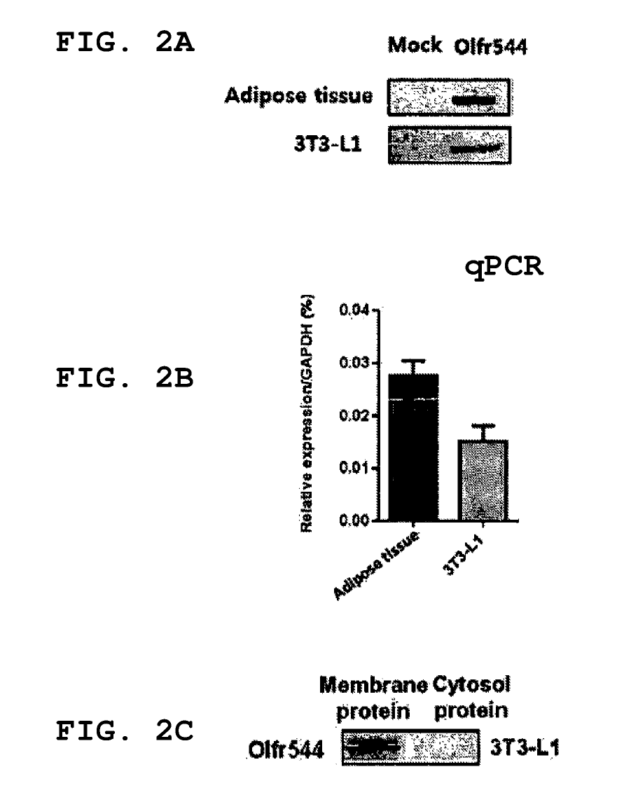

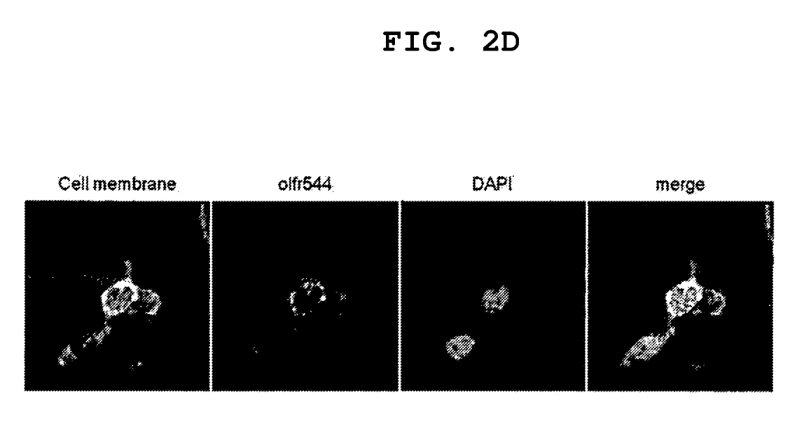

In an example of the present invention, expressions of Olfr544 mRNA and protein in adipose tissue of a mouse and 3T3-L1 adipocytes were analyzed. As shown in FIGS. 2A and 2B, significant RNA expression of the Olfr544 gene was identified in adipose tissue and 3T3-L1 adipocytes by RT-PCR and qPCR experiments. Particularly, both RT-PCR and qPCR experiments showed that the expression level of Olfr544 mRNA was higher in adipose tissue than in 3T3-L1 adipocyte.

In contrast, as shown in FIG. 2C, Olfr544 protein was not detected in the cytosol of 3T3-L1 adipocyte, but was detected at the high level in the membrane protein fraction of 3T3-L1 adipocyte, as analyzed by the western blotting experiment. In addition, as shown in FIG. 2D, the immunohistochemistry experiment showed that Olfr544 protein was significantly expressed in the membrane of 3T3-L1 adipocytes and co-localized with a membrane marker in the membrane of 3T3-L1 adipocytes.

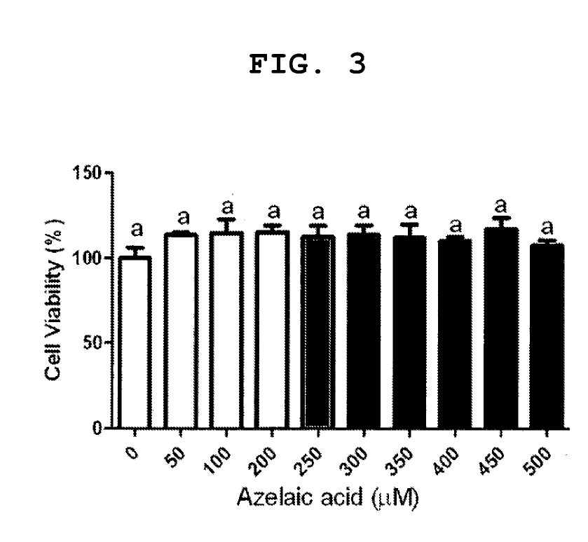

In another example of the present invention, the MTT assay was performed to evaluate the cytotoxicity of azelaic acid. As shown in FIG. 3, cytotoxicity was not observed when 3T3-L1 adipocytes were treated with a high-concentration (500 μM) of azelaic acid.

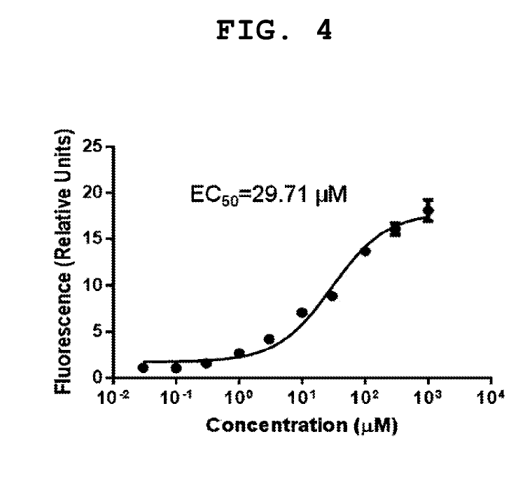

In another example of the present invention, the activity of azelaic acid as an Olfr544 ligand was evaluated. As shown in FIG. 4, azelaic acid increased the activity of Olfr544 within the concentration range of 1 to 1000 μM in a concentration-dependent manner, as analyzed by the cAMP response element (CRE)-luciferase reporter gene assay. The EC₅₀ value was 29.71 μM at which concentration about 50% of Olfr544 was activated.

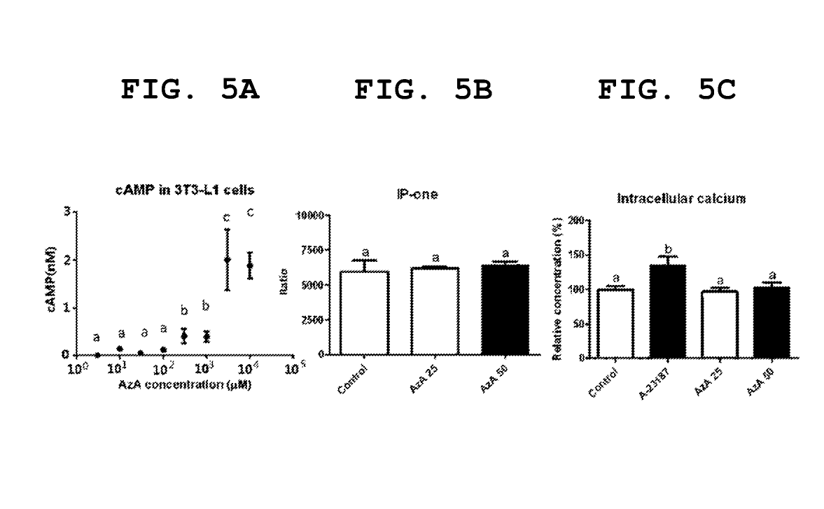

In another example of the present invention, to determine whether azelaic acid involves in signal transduction pathways via activation of Olfr544, the changes of the concentrations of secondary messengers in 3T3-L1 adipocytes upon treatment with azelaic acid were analyzed. As shown FIGS. 5A to 5C, in 3T3-L1 adipocytes treated with azelaic acid, only the concentration of cAMP was specifically increased, proportionally depending on the concentration of azelaic acid.

In another example of the present invention, azelaic acid specifically increased the concentration of cAMP in adipose tissue but did not affect the concentrations of inositol phosphate and calcium (FIG. 5).

Meanwhile, the increased cAMP in adipose cells has been known to induce lipolysis by increasing the CREB activity and stimulating the PKA activity.

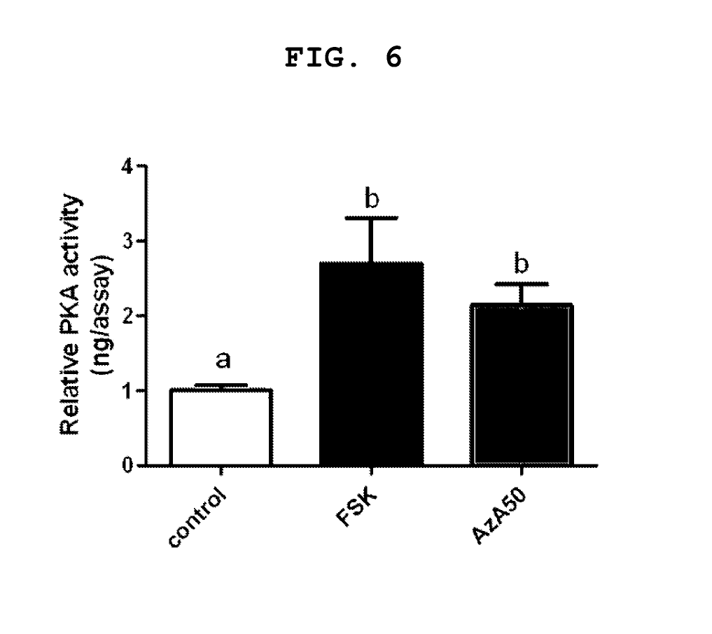

In another example of the present invention, to examine the involvement of the protein kinase A (PKA) signaling pathway, which is the downstream signaling pathway of secondary messenger cAMP, the effect of azelaic acid to regulate the PKA activity was evaluated. As shown in FIG. 6, azelaic acid significantly increased the PKA activity in 3T3-L1 adipocytes. The PKA activity was increased by 113.8% compared to the control when 3T3-L1 adipocytes were treated with azelaic acid (50 μM) for 2 hours. Therefore, the PKA activity was increased due to the increased cAMP in adipocytes by the azelaic acid treatment.

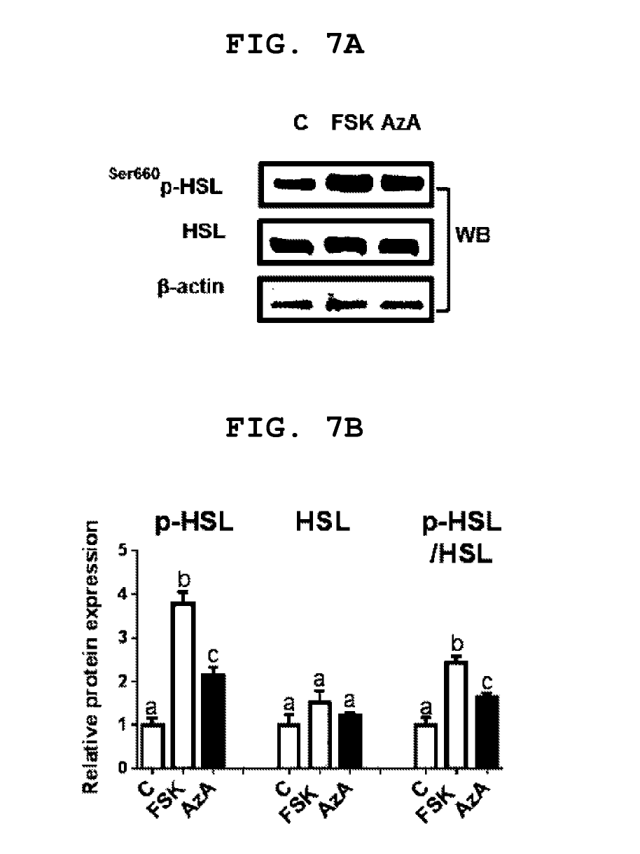

In another example of the present invention, the changes of phosphorylation of cAMP-response element binding protein (CREB) and hormone-sensitive lipase (HSL) protein by azelaic acid were analyzed. As shown in FIG. 7, phosphorylation of CREB and HSL was rapidly increased compared to the control (C) when adipose cells were treated with 50 μM azelaic acid for 2 hours. The increased cAMP in 3T3-L1 adipocytes stimulates the PKA activity, which in turn increases phosphorylation of HSL. Subsequently, activated HSL increases hydrolysis of triglycerides stored in lipid droplets. Accordingly, these results suggest that azelaic acid has an effect of hydrolyzing triglycerides in adipose cells.

In summary, activation of olfactory receptor Olfr544 in adipose tissue (or in adipose cells) by its ligand azelaic acid resulted in activation of PKA via the increased secondary messenger cAMP, which in turn led to phosphorylation and consequent activation of main targets of PKA, CREB and HSL. Accordingly, activation of Olfr544 by azelaic acid leads to activation of the intracellular cAMP-CREB signaling pathway.

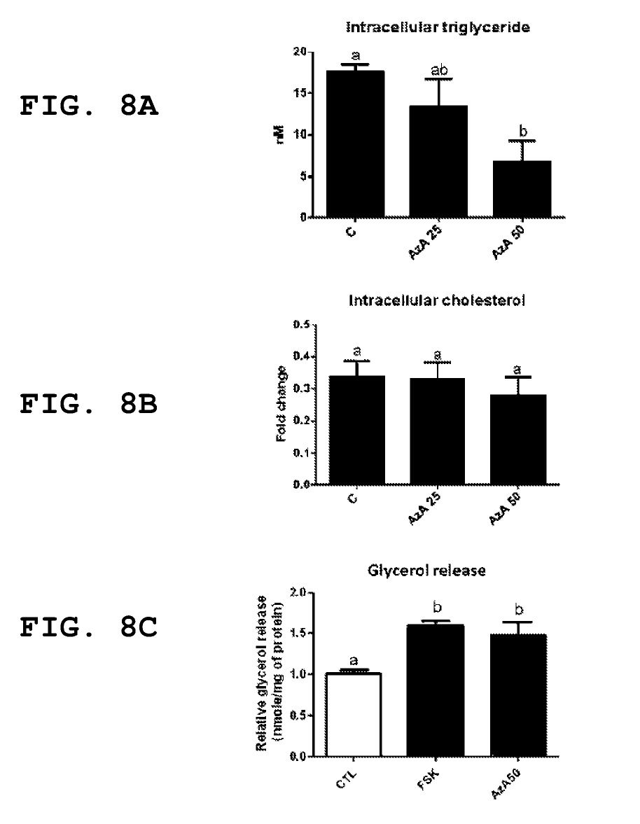

In another example of the present invention, to evaluate the effect of azelaic acid to activate the cAMP-PKA-HSL signaling pathway and resultantly increase triglyceride hydrolysis in 3T3-L1 adipocytes, concentrations of triglycerides and cholesterol in adipose cells and an amount of glycerol released as the product of triglyceride hydrolysis were measured. As shown in FIGS. 8A to 8C, triglycerides in adipose cells were decreased by 61.5% (P<0.05) and glycerol released as the product of triglyceride hydrolysis was increased by 46.7% when 3T3-L1 adipocytes were treated with 50 μM azelaic acid for 2 hours. This indicates that azelaic acid has an effect of increasing triglyceride hydrolysis in adipose tissue and resultantly suppressing accumulation of triglycerides.

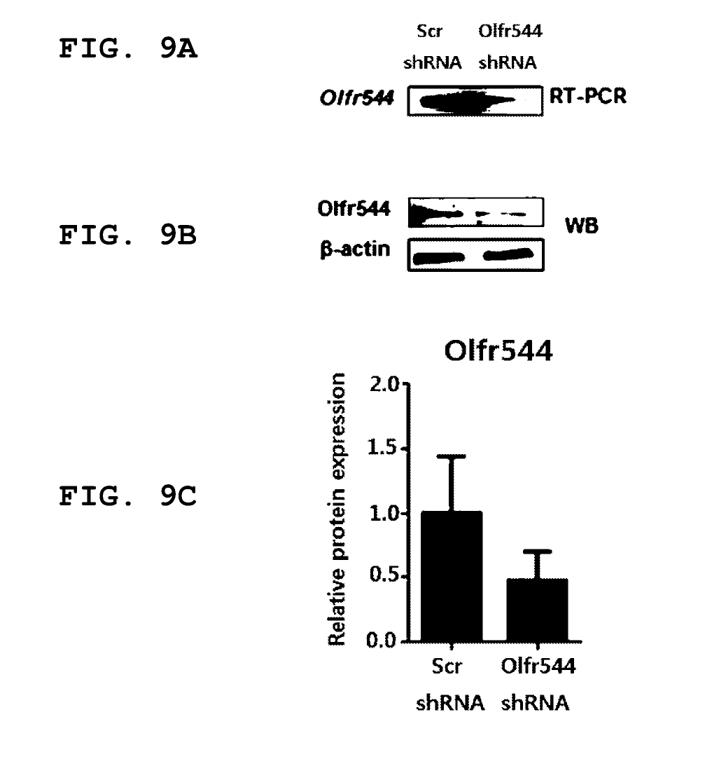

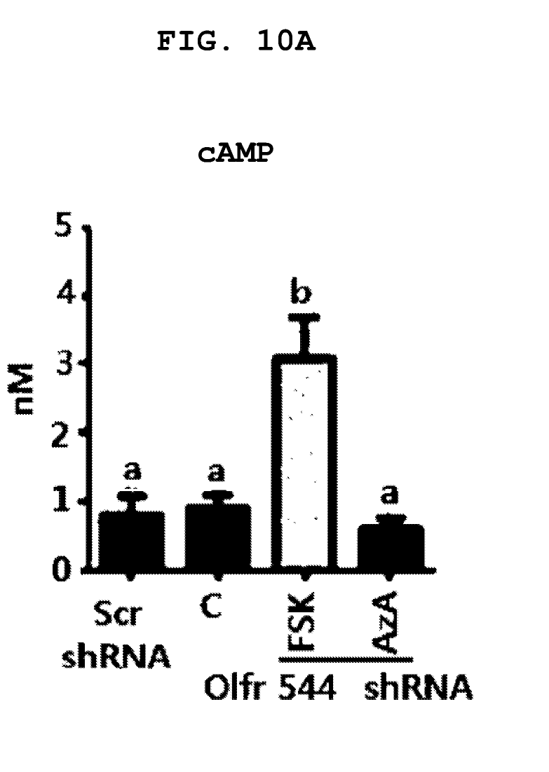

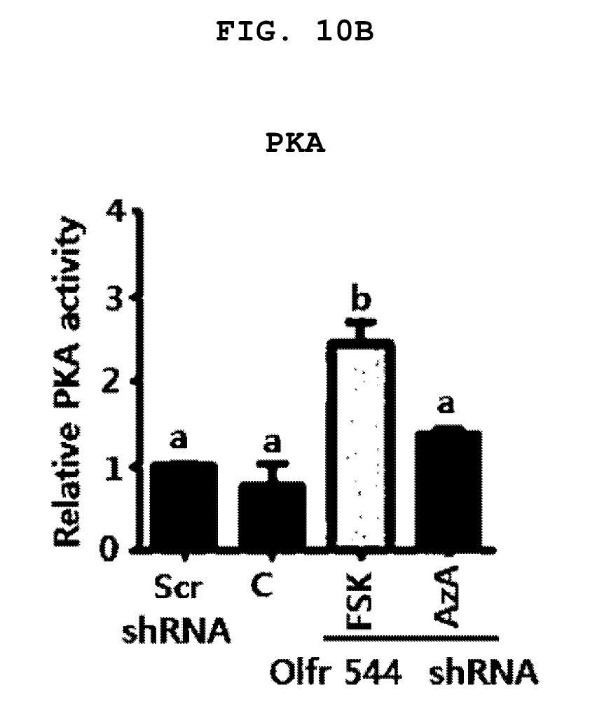

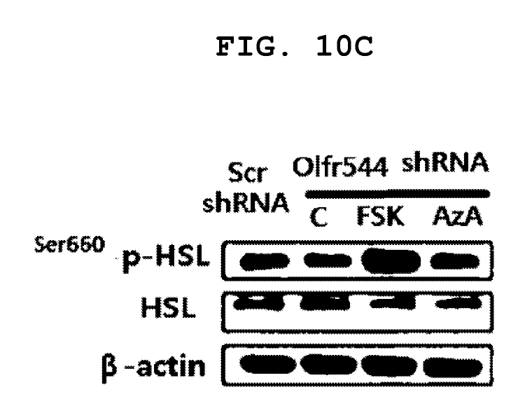

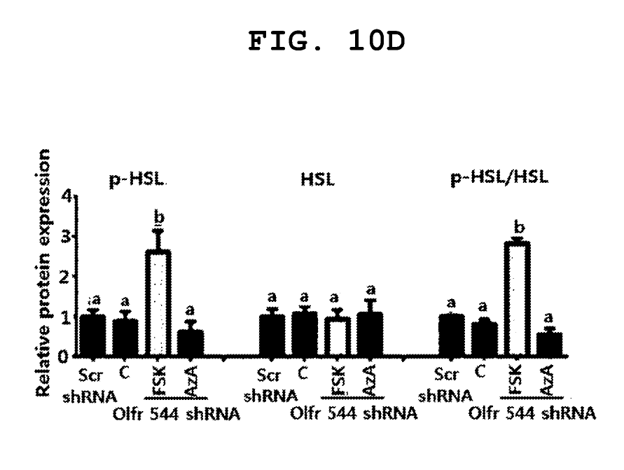

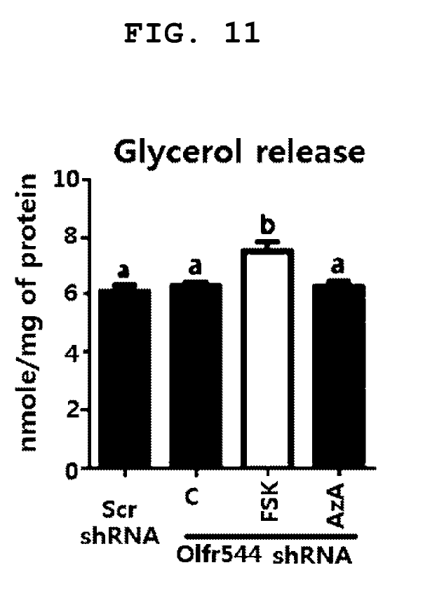

In another example of the present invention, the role of Olfr544 in the azelaic acid-mediated effect of regulating lipolysis was evaluated using an Olfr544 shRNA. Specifically, expression of the Olfr544 gene was suppressed by transfecting an Olfr544 shRNA into mouse 3T3-L1 adipose cells (FIGS. 9A to 9C). In the cells in which expression of Olfr544 was suppressed, the cAMP-PKA-HSL signaling pathway was not activated by the azelaic acid treatment (FIGS. 10A to 10C). Additionally, an increase of glycerol released as the product of triglyceride hydrolysis by the azelaic acid treatment was not observed (FIG. 11). Therefore, these results identified that the effect of azelaic acid to increase lipolysis depends on Olfr544.

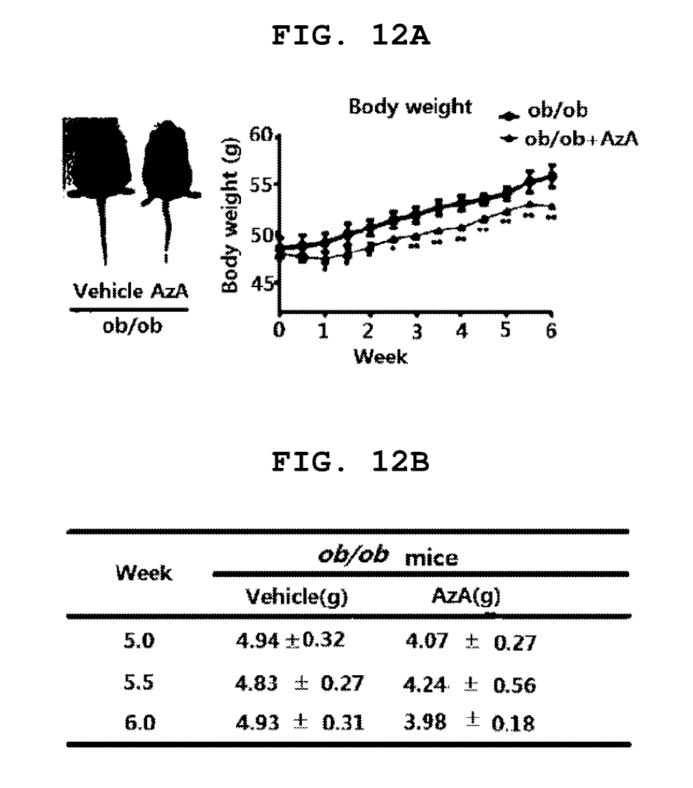

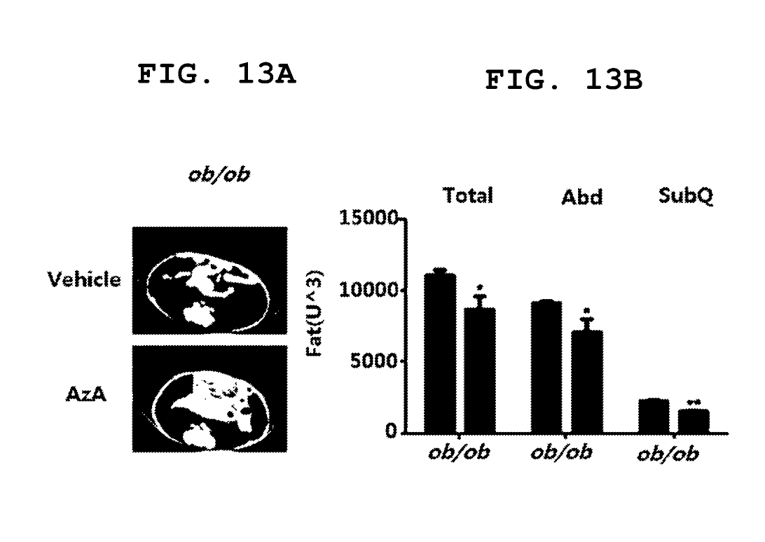

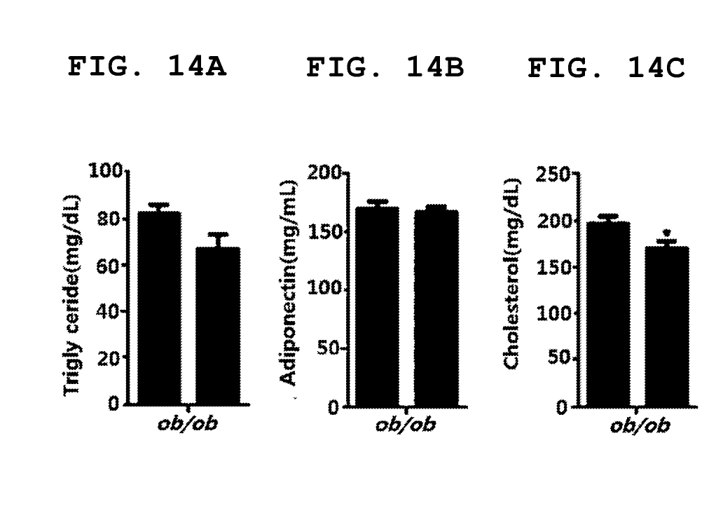

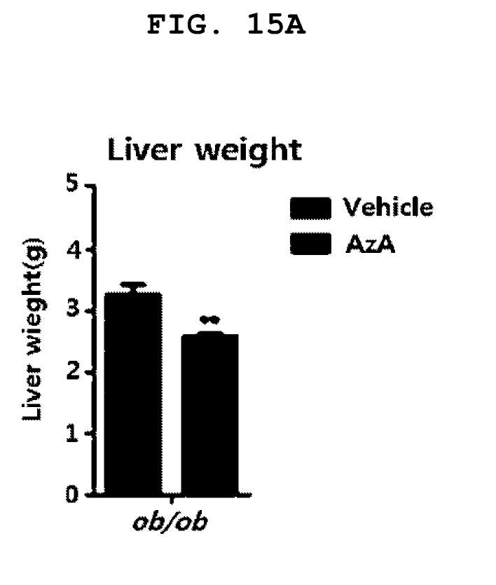

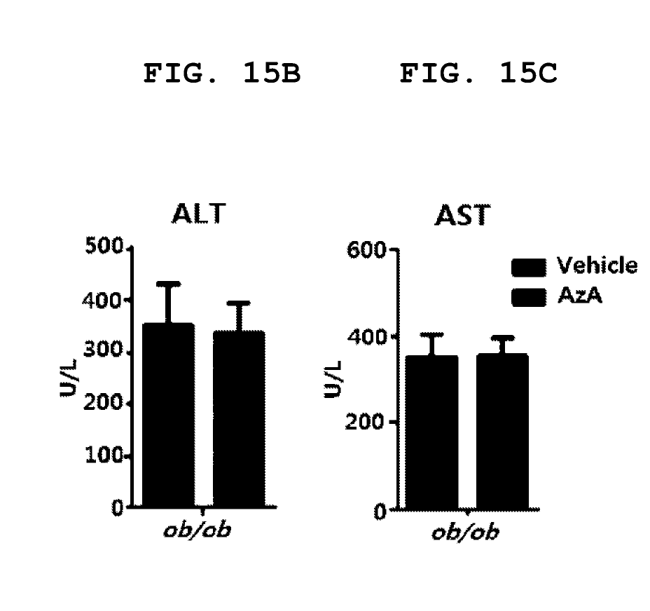

In another example of the present invention, azelaic acid was orally administered to an obesity-induced mouse model and various obesity-associated indices were assessed. When azelaic acid was administered to the obesity mouse group, food intake was not changed, but an increase of body weight was significantly lower compared to the control untreated group (FIG. 12). In addition, total fat, subcutaneous fat, and abdominal fat were significantly decreased compared to the control untreated group (FIG. 13). Furthermore, Upon analysis of blood indices, the blood concentration of cholesterol was significantly decreased by the azelaic acid administration (FIG. 14). In ob/ob mice fed with high-fat diet, the weight of liver tissue was increased due to development of fatty liver. On the contrary, the weight of liver tissue was decreased by administration of azelaic acid, compared to the control untreated group, and side effects, such as hepatotoxicity and the like, as assessed by the level of ALT and AST, were not observed (FIG. 15).

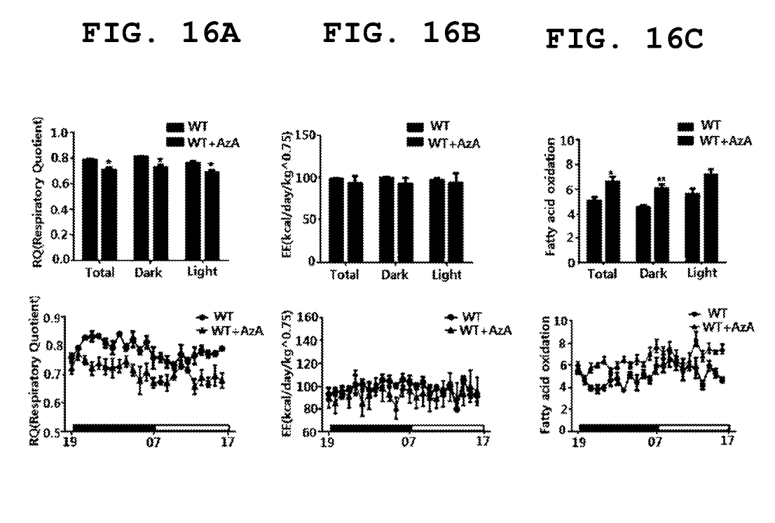

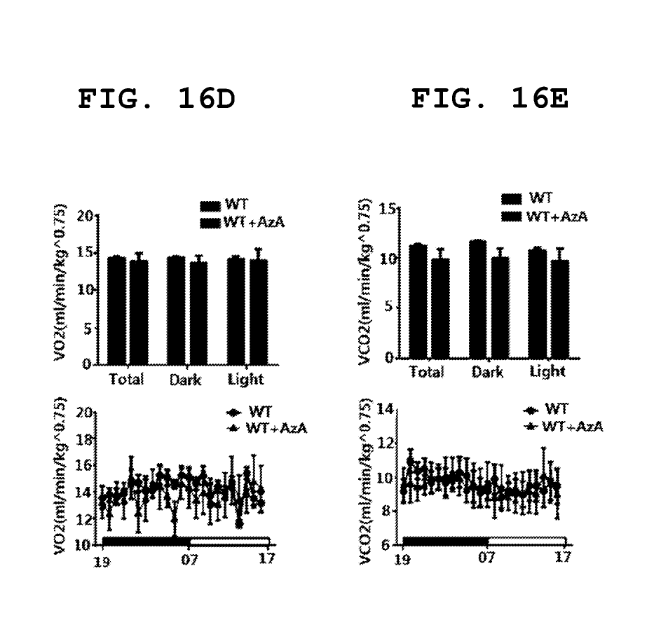

Finally, energy metabolism upon administration of azelaic acid in normal mice was analyzed. In the azelaic acid administration group, while the total energy expenditure (EE) was not changed, the respiratory quotient (RQ) was decreased and fatty acid oxidation was increased compared to the control untreated group. Additionally, no significant difference in the O₂ consumption amount was observed compared to the control untreated group, but the CO₂ production amount was significantly increased compared to the control untreated group (FIG. 16).

In summary, the examples demonstrate that the effect of azelaic acid to increase triglyceride hydrolysis and suppress lipid accumulation in adipose tissue (or in adipose cells) results from the action of azelaic acid as the Olfr544 ligand to activate the intracellular cAMP-PKA-HSL signaling pathway. That is, azelaic acid acts as an agonist of olfactory receptor Olfr544 expressed in the membrane of adipose cells to specifically increase intracellular cAMP, thereby increasing the PKA activity and consequent phosphorylation of CREB and HSL, which in turn increase lipid (triglyceride) hydrolysis. Accordingly, azelaic acid has an effect of suppressing accumulation of triglycerides in adipose tissue, which results in alleviating obesity and metabolic syndromes.

Azelaic acid or a pharmaceutically acceptable salt thereof may be used as a main ingredient or as an additive or an adjuvant to prepare various functional foods and health functional foods.

Therefore, another aspect of the present invention relates to a health functional food composition comprising azelaic acid as an active ingredient for alleviating obesity.

In embodiments of the present invention, alleviating obesity may be characterized by decreasing the level of triglycerides in adipose tissue, suppressing lipid accumulation in adipose tissue, decreasing body weight or decreasing body fat.

In the present invention, the term ‘functional food’ means general food of which functionality is improved by adding azelaic acid. Functionality may be represented by physical properties and physiological functionality. By adding azelaic acid according to the present invention to general food, physical properties and physiological functionality of the general food may be improved. In the present invention, food having improved functions as described above is comprehensively defined as the ‘functional food’.

The functional food according to the present invention may be variously used in drugs, food, beverages and the like, for suppressing accumulation of body fat. Examples of the functional food according to the present invention may include foods, candies, chocolates, beverages, gums, teas, vitamin complexes, health supplement foods and the like, and the functional food may be used in a form of powders, granules, tablets, capsules or beverages.

The composition according to the present invention may be added to food or beverages to increase lipolysis in adipose tissue. The amount of the composition in foods or beverages is as follows: an amount of the composition in the functional food according to the present invention may be generally 0.01 to 50 wt %, preferably 0.1 to 20 wt %, based on a total weight of food, and an amount of the composition in the health beverage according to the present invention may be generally 0.02 to 10 g, preferably 0.3 to 1 g, based on 100 ml of the beverage, but the amounts are not limited to these ranges.

Liquid ingredients of the health beverage according to the present invention are not limited except that the health beverage composition contains the composition according to the present invention as an essential ingredient at the ratio as described above, and the health beverage composition may further contain various flavoring agents, natural carbohydrates and the like, as additional ingredients, similar to general beverages. Examples of the natural carbohydrates include general sugars, for example, monosaccharides, such as glucose, fructose and the like, disaccharides, such as maltose, sucrose and the like, and polysaccharides, such as dextrin, cyclodextrin and the like, and sugar alcohols, such as xylitol, sorbitol, erythritol and the like. In addition to the ingredients described above, as flavoring agents, natural flavors (thaumatin, stevia extracts (for example, rebaudioside A), glycyrrhizin and the like) and synthetic flavors (saccharine, aspartame and the like) may be beneficially used. An amount of the natural carbohydrate in the composition according to the present invention is about 1 to 20 g, preferably about 5 to 12 g, based on 100 ml of the composition.

In addition to the additional ingredients described above, the composition according to the present invention may contain various nutrients, vitamins, minerals (electrolytes), flavorants including synthetic flavorants and natural flavorants, colorants and fillers (cheese, chocolate and the like), pectic acid and salts thereof, alginic acid and salts thereof, organic acids, protective colloid thickeners, pH adjusting agents, stabilizers, preservatives, glycerin, alcohols, carbonating agents used in carbonated beverages and the like. Furthermore, the composition according to the present invention may contain fruit pulps for preparing natural fruit juices, fruit juice beverages and vegetable beverages. These ingredients may be used alone or in combination. Although the amounts of these additives are not particularly important, the amounts are generally selected in a range of 0.01 to 20 parts by weight based on 100 parts by weight of the composition according to the present invention.

### EXAMPLES

Hereinafter, the present invention will be described in more detail in the following examples. However, these Examples are presented solely for the purpose of illustrating the present invention, and those skilled in the art will appreciate that these examples are not to be construed as limiting a scope of the present invention.

### Example 1: Analysis of Olfr544 Gene and Protein Expression, Molecular Target of Azelaic Acid

To analyze expression of olfactory receptor Olfr544 (hereinafter referred as Olfr544) protein, a molecular target of azelaic acid in adipose cells, experiments for measuring expression of mRNA and protein were performed (FIG. 2). Expression of Olfr544 mRNA in mouse adipose tissue and differentiated 3T3-L1 adipocytes was analyzed using RT-PCR and quantitative real time (qPCR) experiments, respectively, and expression of Olfr544 protein in the cells was analyzed using western blotting and immunohistochemistry experiments.

### 1-1: Cell Culture

Adipose cells used in Examples 1 to 7, which were developed by differentiating 3T3-L1 preadipocytes (KCLB No. 10092.1) were obtained from Korean Cell Line Bank. These cells were grown in a Dulbecco's modified Eagle's medium (DMEM) containing 10% fetal calf serum (FCS) at 37□ under 5% CO₂ condition, sub-cultured at a confluency (the level of proliferation) of 50%, and maintained (Green H., et al., Cell, 3(2):127-33, 1974; Green H., et al., Cell, 5(1):19-27, 1975; Atanasov, A. G., et al., Biochimica et Biophysica Acta(BBA)-General Subjects 1830(10):4813-9).

### 1-2: Cell Differentiation

The differentiation process of 3T3-L1 preadipocytes was as follows. First, 1×10⁶ 3T3-L1 preadipocytes were seeded in each well of a 6-well plate and cultured to a confluency of 100%. After 2 days of culture, the cultured 3T3-L1 preadipocytes were treated with the DMEM containing 10% fetal bovine serum (FBS) and an MDI solution (0.5 mM IBMX, 0.5 μM dexamethasone, and 10 μg/mL insulin) for 3 days. The treated cells were cultured in the DMEM containing 10% FBS and 10 μg/mL insulin. Differentiation of adipocytes were adjusted according to appearance of lipid droplets in the cells (Green H., et al., Cell, 3(2):127-33, 1974; Green H., et al., Cell, 5(1):19-27, 1975; Atanasov, A. G., et al., Biochimica et Biophysica Acta(BBA)-General Subjects 1830(10):4813-9).

Resultantly, at least about 80% of 3T3-L1 preadipocytes were differentiated into adipocytes through the differentiation process for 12 days (data not shown).

1-3: Expression of Olfr544 mRNA

To examine mRNA expression of Olfr544, RT-PCR and qRT-PCR experiments were performed.

(1) Synthesis of cDNA

First, RNA was extracted from 3T3-L1 adipocytes differentiated as described in Examples 1-1 and 1-2 using the method known to those skilled in the art, and cDNA was synthesized using the extracted RNA by the ReverTra Ace® qPCR RT kit (TOYOBO, Osaka, Japan). More specifically, to improve the efficiency of the RT-PCR reaction, the extracted RNA was treated at 65□ for 5 minutes and immediately stored in ice. Total 8 μl of a reactant was prepared by mixing 2 μl of 4×DNA Master Mix containing a gDNA remover, 0.5 μg of RNA and distilled water (nuclease-free water). The reactant was incubated at 37□ for 5 minutes, followed by addition of 5×RT Master mix to the product. The reaction to synthesis cDNA was performed at 37□ for 5 minutes, at 50□ for 5 minutes, and at 98□ for 5 minutes.

(2) PCR

A polymerase chain reaction (PCR) was performed using the cDNA synthesized as described in (1) using the primers shown in Table 1 by the conventional methods known to those skilled in the art. The resulting PCR products were analyzed by electrophoresis on agarose gel, and expression of Olfr544 mRNA was examined by appearance of a corresponding band on the gel. Glyceraldehyde-3-phosphate dehydrogenase (GAPDH, a sequence generally known to those skilled in the art) was used as a control.

The following Table 1 shows the Olfr544 primer sequences.

TABLE 1

| | | | SEQ ID |
|---|---|---|---|
| Name | type | Sequence (5′->3′) | NO: |
| mOlfr544_F | sense | GGG GAC ATC TCG CTG AAT | 1 |
| | | AA | |
| mOlfr544_R | anti-sense | ATG AGG ACA TGG TGG AGG | 2 |
| | | AG | |

(3) qPCR (Quantitative Real-Time PCR)

qPCR was performed using the cDNA synthesized as described in (1), the primers shown in Table 1 and the Thunderbird Sybr qPCR Mix (TOYOBO, Osaka, Japan) with the iQ5 iCycler system (Bio-Rad, California, U.S.) according to the methods known to those skilled in the art. More specifically, a denaturation process was performed at 95□ for 4 minutes and 30 seconds, followed by 40 cycles of the reaction composed of a denaturation process at 95□ for 10 seconds, an annealing process at 55□ to 60□ for 30 seconds, and an extension process at 68□ for 20 seconds. The expression level of each mRNA was standardized using the expression level of GAPDH mRNA as a control to compare. The primers were designed using the nucleotide Blast software of National Center for Biotechnology Information (NCBI) and purchased from Bionics (Seoul, Korea).

The results shown in FIGS. 2A and 2B demonstrate that the Olfr544 gene was significantly expressed at the mRNA level in mouse adipose tissue and 3T3-L1 adipocytes as analyzed by the RT-PCR and qPCR experiments. Particularly, in RT-PCR and qPCR experiments, the expression level of Olfr544 in mouse adipose tissue was higher than the level in 3T3-L1 adipocytes.

### 1-4: Expression of Olfr544 Protein

To analyze expression of Olfr544 protein, the final product of gene expression, protein electrophoresis (sodium dodecyl sulfate-polyacrylamide gel electrophoresis (SDS-PAGE)), western blotting and immunohistochemistry experiments were performed.

(1) Protein Extraction

Proteins were extracted from 3T3-L1 adipocytes differentiated as described in Examples 1-1 and 1-2.

To obtain the membrane protein fraction, 3T3-L1 adipocytes (or adipose tissue) were rinsed with phosphate-buffered saline (PBS) and only cell (or tissue) pellets were collected. The collected cell (or tissue) pellets were washed with Solution C (120 mM NaCl, 5 mM KCl, 1.6 mM MgSO₄, 25 mM NaHCO₃, 7.5 mM D-glucose, pH 7.4). Solution D (the solution C+10 mM CaCl₂) was added to the washed cell (tissue) pellets and the resulting cell (tissue) mixture was stirred for 20 minutes, followed by centrifugation. The supernatant after centrifugation was re-centrifuged and the precipitate was collected after the second centrifuge. The collected precipitate was dissolved in the TEM buffer solution (10 mM Tris, 3 mM MgCl₂, 2 mM EDTA, pH 8.0) containing glycerol, thereby obtaining the membrane protein fraction. The protein extraction method described above is known as the calcium ion shock method, wherein the protein yield is higher than that obtained by the mechanical stirring method, a conventional membrane fractionation method.

To obtain the cytosol protein fraction, 3T3-L1 adipocytes (or adipose tissue) were rinsed with PBS and only cell (tissue) pellets were collected. The digitonin-containing buffer solution (150 mM NaCl, 50 mM HEPES, 25 μg/ml digitonin, pH 7.4) was added to the collected cell (tissue pellets). The resultant mixture was incubated on ice for 10 minutes, followed by centrifugation. The supernatant obtained after centrifugation was the cytosol protein fraction.

To obtain the whole protein extract without fractionation processes, the cell lysis buffer solution (50 mM Tris, 1% Triton-X 100, 1 mM EDTA, proteinase inhibitor (PI)) was added to 3T3-L1 adipocytes to lyse cells, followed by centrifugation at 13,000 rpm at 4□ for 20 minutes. The supernatant obtained after the centrifugation is the whole protein extract.

(2) Protein Electrophoresis (SDS-PAGE) and Western Blot

To analyze the Olfr544 protein expression, the protein fractions (membrane protein fraction, cytosol protein fraction and whole protein extract) described (1) were quantified by the Bradford method. About 40 μg of protein from each fraction sample was aliquoted and protein fraction was denatured by suitably heating and/or dissolving the protein extract depending on the type of the extracted protein (fractionated protein, whole protein, or phosphorylated protein and the like). The denature protein extract was analyzed by 12% SDS-PAGE.

The western blotting experiment was performed as follows: the protein on the SDS-PAGE gel was transferred to nitrocellulose (NC) membrane. The NC membrane was blocked to prevent non-specific binding. After blocking, the membrane was treated with a primary antibody (anti-Olfr544 rabbit antibody (Abcam, U.K.)) to detect the protein, followed by the treatment with a secondary antibody (HRP-conjugated-anti-rabbit IgG, Santa Cruz, Calif., U.S.), at room temperature for 1 hour, respectively. Anti-β-actin antibody (Santa Cruz, Calif., U.S.) was used to measure the β-actin expression as a loading control. To compare the quantity of each protein band in the western blotting experiment, protein bands were analyzed and quantified by the Gel-Pro Analyzer 4.0 software program.

(3) Immunohistochemistry

To analyze the intracellular expression and sub-cellular location of Olfr544 by immunohistochemistry, cultured 3T3-L1 adipocytes were seeded in a 6-well plate at a concentration of 1×10⁵ cells/well and incubated for 24 hours for attachment and stabilization. A lucy-flag-Olfr544 expression vector was transfected into 3T3-L1 adipocytes using Lipofectamine 2000 (Invitrogen, California, U.S.). After 24 hours, the 3T3-L1 adipocytes transfected with the lucy-flag-Olfr544 expression vector were washed with PBS and fixed with 4% para-formaldehyde for 20 minutes. Olfr544 protein, the membrane, and the nucleus were stained with dyes (i, ii and iii), respectively, as described below.

i) To stain Olfr544 protein, 3T3-L1 adipocytes were incubated with an anti-flag primary antibody (Sigma, U.S.) or an anti-Olfr544 antibody (Abcam, Cambridge, U.K.) at room temperature for 1 hour and washed with PBS three times, followed by incubation with an anti-rabbit IgG secondary antibody labeled with green fluorescence for 1 hour. During the incubation with the secondary antibody, light was blocked using foil.

ii) To stain the membrane, 3T3-L1 adipocytes were treated with the fluorescence-labeled CellMask™ Orange Plasma Membrane Stains (Invitrogen, California, U.S.) at 37□ for 10 minutes.

iii) To stain the nucleus, 3T3-L1 adipocytes were treated with the 4′,6-diamidino-2-phenylindole (DAPI) solution (Sigma, U.S.). After staining, a cover slide was placed thereon to airtightly seal the stained nucleus.

The stained 3T3-L1 adipocytes as described above were photographed using a confocal fluorescent microscope (LSM700, Carl Zeiss).

As shown in FIG. 2C, Olfr544 protein was not detected in the cytosol of 3T3-L1 adipocytes, but was detected at the high level in the membrane protein fraction, as identified by the western blotting experiment. In addition, as shown in FIG. 2D, the immunohistochemistry experiment demonstrated that the Olfr544 protein was significantly expressed in the membrane and co-localized with a membrane marker in the membrane of the 3T3-L1 adipocyte.

### Example 2: Evaluation of the Cytotoxicity of Azelaic Acid

To evaluate the cytotoxicity of azelaic acid, the MTT assay was performed.

The MTT assay is a test method based on the ability of mitochondria to reduce MTT tetrazolium, a yellow water-soluble substrate, to a blue purple water-insoluble MTT formazane (3-(4,5-dimethylthiazol-2-yl)-2,5-diphenyl-tetrazolium bromide). An MTT reagent was prepared by diluting C₁₈H₁₆BrN₅S (Thiazoly Blue Tetrazolium Bromide) in phosphate buffered saline (PBS) at a concentration of 2 to 5 mg/ml.

First, 3T3-L1 adipocytes (4×10⁵ cells/ml) were seeded in a 96-well plate and cultured at 37□ under 5% CO₂ condition for 24 hours. The cultured 3T3-L1 adipocytes were treated with azelaic acid in a concentration range of 0 to 500 μM and incubated for 24 hours. 100 μL of the MTT reagent was added to treated 3T3-L1 adipocytes at a concentration of 4 mg/mL and incubated at 37□ under 5% CO₂ condition for 4 hours. 100 μL of dimethyl sulfoxide (DMSO) was added thereto and absorbance was measured at 540 nm. As the absorbance is in proportion to the number of living cells, the absorbance value is used to quantify the apoptotic effect due to the cytotoxicity.

As shown in FIG. 3, cytotoxicity was not observed when 3T3-L1 adipocytes were treated with a high-concentration (500 μM) of azelaic acid. Therefore, azelaic acid contained in various edible plants, including grains, such as barley, oats and the like, and produced during a metabolic process in the human body, thereby present in a concentration range of about 10 to 50 μM, may be safely ingested.

### Example 3: Evaluation of the Activity of Azelaic Acid as Olfr544 Ligand

To evaluate the activity of azelaic acid as an Olfr544 ligand in vitro assay, the reporter gene assay was performed. Generally, the gene expression level of olfactory receptors (OR) in tissue cells is low and the protein expression level in the cell membrane is also low, such that research on olfactory receptors is difficult. To overcome this problem, the Hana3A cell line provided by professor Hiroaki Matsunami's research team at Duke University (Zhuang H., et al., Nat. Protocol, 3:1402-1413, 2008) was used in experiments. The Hana3A cell line is a stable cell line generated by transforming the HEK293T cell line to co-express the receptor-transporting protein 1S (RTP1S) and the RIC8 Guanine Nucleotide Exchange Factor B (Ric8b), which are essential cofactors for membrane expression of OR proteins, and thus significantly improving (optimizing) membrane expression of OR proteins.

The reporter gene assay was performed as follows. First, an Olfr544 expression vector and a cAMP response element (CRE)-Luciferase reporter vector were transfected into the Hana3A cell line. The transfected Hana3A cell line was treated with azelaic acid for 18 hours. The luciferase activity was measured using the Firefly luciferase (FL) assay kit. The dose response curve (DRC) was deduced based on the measured luciferase activity and the 50% effective concentration (EC₅₀) was calculated as an index to determine the activity as a ligand (agonist).

As shown in FIG. 4, azelaic acid increased the activity of Olfr544 within the concentration range of 1 to 1000 μM in a concentration-dependent manner, as analyzed by the cAMP response element (CRE)-luciferase reporter gene assay. The EC₅₀ value was 29.71 μM at which concentration about 50% of Olfr544 was activated. Azelaic acid is naturally produced in the body, and generally, the blood concentration of azelaic acid has been variously reported at several tens to several hundreds micromolar (μM) levels. Hence, the measured EC₅₀ value indicates that azelaic acid may activate Olfr544 in the body without inducing toxicity.

Subsequently, an effective administration dose of azelaic acid was calculated based on the EC₅₀ value (29.71 μM) of azelaic acid in connection with Olfr544, a target protein of azelaic acid, as shown in FIG. 4, and the EC₅₀ value of Actos® (pioglitazone, PPAR-γ agonist, EC₅₀=0.5 μM, human administration dose: 15-30 mg/day), a drug for metabolic diseases, and the EC₅₀ value of Xenical® (orlistat, pancreatic lipase inhibitor, EC₅₀=0.24 μM, human administration dose: 120 mg/day). The effective administration dose of azelaic acid calculated is about 900 to 1800 mg/day. This is a dose usable as a daily intake amount, and considering an amount of azelaic acid produced in the body and an amount of azelaic acid ingested from general food, an effective intake amount of azelaic acid may be further decreased.

### Example 4: Analysis of the Concentration Changes of Secondary Messengers in 3T3-L1 Adipocytes Treated with Azelaic Acid

To examine whether azelaic acid activates Olfr544, a G-protein coupled receptor (GPCR), and participates in signal transduction pathways associated with triglyceride hydrolysis.

GPCR activates G-proteins by ligand binding and consequently regulates intracellular signal transduction using secondary messengers. Depending on the type of G-protein, cAMP, IP₃ and/or calcium are used as secondary messengers.

The Changes of the concentrations of cAMP, IP-one (metabolite of IP₃) and calcium, which may be secondary messengers of azelaic acid as Olfr544 ligand, were measured in 3T3-L1 adipocytes. Since IP₃ has a low stability and is easily metabolized to IP-one, the amount of intracellular IP-One (metabolite of IP₃) was quantified instead.

cAMP assay is designed to measure the amount of cAMP produced as a secondary messenger of a GPCR receptor upon activation by a ligand using the cAMP standard calibration curve. The 96-well plate provided in the cAMP assay ELIS kit (Enzo Life science, New York, U.S.) is coated with a GxR IgG antibody. Once cAMP of the blue solution provided for obtaining the standard calibration curve binds to alkaline phosphatase, the color of the blue solution is changed to yellow by a rabbit antibody. This is because alkaline phosphatase is activated by adding the pNpp substrate to the cAMP-bound well and the color is change. The concentration of cAMP can be measured at the wavelength of 405 nm using the colorimetric analysis. In the present experiment, 1×10⁴ 3T3-L1 adipocytes were seeded in each well of the 96-well plate, differentiated, and then treated with 50 μM azelaic acid and 1 μM Foskolin as a positive control, respectively, for 18 hours. The concentration of cAMP was measured as described above.

IP-one assay is designed to measure the concentration of IP₃, secondary messenger formed by phospholipolysis by phospholipase in a GPCR signal transduction process and thus assess the ability of regulating the protein kinase C (PKC) signal transduction pathway. In the present experiment, 1×10⁴ 3T3-L1 adipocytes were seeded in each well of the 96-well plate, differentiated, and then treated with 50 μM azelaic acid for 18 hours. To measure the concentration of IP₃, the concentration of intracellular IP-one (metabolite of IP₃, a surrogate marker) was measured using an IP-one ELISA Assay kit (Cisbio, France), instead, as the error in measuring the concentration of IP-one was smaller than that of IP₃.

The concentration of intracellular calcium was measured using the Fluo-4 Direct™ Calcium Assay Kit (Invitrogen, California, U.S.) containing a fluorescent dye for labeling calcium ions. 3T3-L1 adipocytes were cultured, collected and subjected to centrifugation. Only pellets were collected after centrifugation and diluted in the Fluo-4 Direct™ calcium assay buffer solution provided in the kit at a concentration of 2.5×10⁶ cells/mL. The resulting solution was placed in a 96-well plate and incubated at 37□ under 5% CO₂ condition for 60 minutes. 50 μL of the 2×Fluo-4 Direct™ calcium reagent loading solution was added to the 96-well plate and incubated at 37□ under 5% CO₂ condition for 30 minutes. Fluorescence was measured at excitation and emission wavelengths of 494 nm and 516 nm.

As shown in FIGS. 5A to 5C, azelaic acid significantly increased the concentration of intracellular cAMP but did not affect the concentrations of IP₃ and calcium. The increased cAMP concentration by azelaic acid appear to depend on the concentration of azelaic acid. Therefore, azelaic acid specifically increases the concentration of intracellular cAMP by activating Olfr544 in 3T3-L1 adipocytes.

### Example 5: Evaluation of the Effect of Azelaic Acid on Regulating the PKA Activity

Protein kinase A (PKA) is a representative protein that regulates the protein activities associated with the signal transduction pathway depending on the change of the concentration of intracellular cAMP in the tissue cells. Under normal conditions, the PKA activity is suppressed as the enzyme active site is blocked by the regulatory domain site of the protein. However, when the concentration of cAMP is increased, cAMP binds the regulatory site of PKA. Consequently the active site is exposed and the PKA activity is increased. In the present experiment, the change of the PKA activity by azelaic acid was analyzed. The PKA activity was measured using the Enzo PKA kinase activity kit (Cat. # ADI-EKS-390A, Enzo Life Sciences, Korea).

As shown in FIG. 6, azelaic acid significantly increased the PKA activity in 3T3-L1 adipocytes. The PKA activity was increased by 113.8% compared to the control when 3T3-L1 adipocytes were treated with azelaic acid (50 μM) for 2 hours. Therefore, the PKA activity was increased due to the increased cAMP in adipocytes by azelaic acid treatment.

### Example 6: Analysis of the Changes of Phosphorylation of CREB and HSL by Azelaic Acid

When the PKA protein activity is increased by azelaic acid, phosphorylation of target proteins of PKA is increased. Therefore, phosphorylation of cAMP-response element binding protein (CREB) and hormone-sensitive lipase (HSL), which are known as target proteins of PKA, was analyzed by the western blotting experiment. In the present western blotting experiment, the method described in Example 1 was used. After 3T3-L1 adipocytes were treated with azelaic acid for 2 hours, proteins were extracted and the experiment was performed. Primary antibodies used in the present experiment were anti-phospho-CREB and anti-phospho-HSL antibodies (Santa Cruz, Calif., US).

As shown in FIG. 7, phosphorylation of CREB and HSL was rapidly increased compared to the control (C) when adipose cells were treated with 50 μM azelaic acid for 2 hours. The increased cAMP in adipocytes stimulates the PKA activity, which in turn increases phosphorylation of HSL.

Subsequently, activated HSL increases hydrolysis of triglycerides stored in lipid droplets. Therefore, these results suggest that azelaic acid has an effect of hydrolyzing triglycerides in adipocytes.

### Example 7: Analysis of the Changes of the Concentrations of Triglyceride, Cholesterol and Lipid Hydrolysis in Adipocytes by Azelaic Acid

To analyze whether azelaic acid induces triglyceride hydrolysis in adipocytes by activating the cAMP-PKA-HSL signaling pathway, adipocytes were treated with azelaic acid and concentrations of intracellular lipids were measured.

After treating 3T3-L1 adipocytes with azelaic acid (concentration: 25 or 50 μM), intracellular lipids were extracted. The concentration of triglycerides was measured using the TG assay kit (BioVision, California, US) and the concentration of cholesterol was measured using the Amplex Red cholesterol assay kit (Invitrogen, California, US). As shown in FIGS. 8A to 8C, azelaic acid significantly decreased the concentration of triglycerides in a concentration-dependent manner in 3T3-L1 adipocytes. The concentration of intracellular triglycerides was decreased by 61.5% compared to the control when 3T3-L1 adipocytes were treated with 50 μM azelaic acid. In contrast, the concentration of intracellular cholesterol was not significantly changed. Therefore, azelaic acid selectively hydrolyzed triglycerides in adipose tissue.

In addition, to analyze whether azelaic acid has an effect of hydrolyzing triglycerides, the amount of glycerol released as the product of triglyceride hydrolysis was measured. The amount of glycerol was increased by 46.7% compared to the control, exhibiting significant increase of the amount of glycerol released as the product of triglyceride hydrolysis when the 3T3-L1 adipocytes were treated with 50 μM azelaic acid for 2 hours.

Taken together, azelaic acid according to the present invention binds Olfr544, GPCR expressed in the cell membrane of the adipocytes, as a ligand, and activates Olfr544, which in turn activates the cAMP-PKA-HSL signaling pathway, thereby hydrolyzing triglycerides to decrease the concentration of triglycerides in adipocytes. Particularly, as azelaic acid is a natural product produced in the body and is contained in grains, such as barley, rye and the like, azelaic acid is an edible ingredient that acts on adipocytes in the body to increase triglyceride hydrolysis, thereby allowing the effect of decreasing a body weight and the anti-obesity effect.

### Example 8: Analysis of the Olfr544 Dependency in the Effect of Azelaic Acid to Regulate Lipolysis Using Olfr544 shRNA

8-1: Knock-Down of Olfr544 Gene in Adipocytes Using Olfr544 shRNA

To analyze the knock-down effect of Olfr544 in mouse adipocytes, 3T3-L1 cells were differentiated into adipocytes in a 6-well plate. The shRNA sequence targeting Olfr544 (top strand: 5′-CACCGCTCACTGTTCGCATCTTCATTCGAAAATGAAGATGCGAACAGTGAG-3′: SEQ ID NO: 3) or encoding non-targeting scrambled shRNA hairpins (top strand: 5′-CACCGTAAGGCT-ATGAAGAGATACCGAAGTATCTCTTCATAGCCTTA-3′: SEQ ID NO: 4) was inserted into the pENTR/U6 vector using the Block-iT U6 RNAi entry vector kit (Invitrogen) to construct an Olfr544 shRNA. 2.5 μg of Olfr544 shRNA or scrambled shRNA was transfected into 3T3-L1 cells using 10 μL of Lipofectamin2000 (Invitrogen). After 48 hours, Olfr544 expression was measured. The expression levels of Olfr544 mRNA and protein were analyzed by the methods described in Examples 1-3 and 1-4. Scr shRNA was used as a negative control.

As shown in in FIGS. 9A to 9C, the expression levels of Olfr544 mRNA and protein was decreased by 80% and 50%, respectively, by the Olfr544 shRNA.

8-2: Effect of Inactivating Signal Transduction by Azelaic Acid in Adipocyte in which the Olfr544 Gene Expression was Knocked-Down

After the Olfr544 gene expression was knocked-down in mouse adipocytes, the cAMP assay was performed by the method described in Example 4, the PKA activity was measured by the method described in Example 5, and phosphorylation of HSL protein was analyzed by the method described in Example 6.

As shown in FIGS. 10A to 10C, in the adipocytes to which the Olfr544 shRNA was transfected and thus the Olfr544 gene expression was knocked-down, the cAMP-PKA-HSL signaling pathway remained inactive even after the adipocytes were treated with azelaic acid.

8-3: Measurement of the Effect of Azelaic Acid on Lipolysis in Adipocyte in which the Olfr544 Gene Expression was Knocked-Down

After the Olfr544 gene expression was knocked-down in mouse adipocytes, the adipocytes were treated with azelaic acid and whether the effect of azelaic acid on triglyceride hydrolysis was maintained was analyzed.

As shown in FIG. 11, no changes of the concentration of glycerol were observed in the adipocytes where the Olfr544 shRNA was transfected and thus the Olfr544 gene expression was knocked-down even after the adipocytes were treated with azelaic acid. Therefore, the effect of azelaic acid on lipolysis in adipocytes was dependent on Olfr544.

### Example 9: Analysis of the Anti-Obesity Effect of Azelaic Acid Using Obesity-Induced Mouse Model

The development of obesity was compared between the genetically obesity-induced ob/ob mouse (Samtako, Seoul, Korea) to which azelaic acid was orally administered at a concentration of 50 mg/kg and those of the control group to which azelaic acid was not administered. More specifically, the increase of body weight, the feed intake amount, the body fat amount, the blood indices, the blood lipid indices, the weight of liver tissue, the hepatotoxicity indices, including ALT and AST, and the energy metabolism level were analyzed.

9-1: Analysis of the Increase of Body Weight and the Feed Intake Amount after Administration of Azelaic Acid

The changes of body weight of the azelaic acid-administered group and the control group were observed once a week for 6 weeks.

As shown in FIGS. 12A and 12B, the body weight of the azelaic acid-administered group was significantly decreased on and after the first week of the experiment compared to the control group. Afterward, the increase of body weight of the azelaic acid-administered group was smaller than the control group. Meanwhile, no significant difference in the feed intake amount was observed between the azelaic acid-administered group and the control group when measured at the final week of the experiment.

9-2: Analysis of Body Fat after Administration of Azelaic Acid

Body fats (total fat, subcutaneous fat, and abdominal fat) of mice in the azelaic acid-administered group to which azelaic acid was orally administered for 6 weeks and the control group were measured using the micro CT image analysis. The subcutaneous fat was quantified for the entire body of the mouse, and the abdominal fat was calculated by quantifying the amount of abdominal fat at the 15th to 20th lumbar spine segments of the mouse.

As shown in FIG. 13, the total fat, the subcutaneous fat and the abdominal fat of the mice to which azelaic acid was orally administered for 6 weeks were significantly decreased compared to the control group.

9-3: Analysis of the Blood Lipid Indices after Administration of Azelaic Acid

To evaluate the effect of azelaic acid on mouse lipid metabolism in the azelaic acid-administered group to which azelaic acid was orally administered for 6 weeks and the control group, the concentrations of blood triglycerides, adiponectin and cholesterol were measured.

As shown in FIG. 14, in the azelaic acid-administered group, the concentrations of blood triglyceride and cholesterol were significantly decreased.

9-4: Analysis of the Weight of Liver Tissue, ALT and AST after Administration of Azelaic Acid

The weight of liver tissue, ALT and AST of the mice in the azelaic acid-administered group to which azelaic acid was orally administered for 6 weeks and the control group were measured.

As shown in FIGS. 15A to 15C, the weight of liver tissue was decreased in the azelaic acid-administered group, but no big difference of the hepatotoxicity indices, ALT and AST, were observed between the azelaic acid-administered group and the control group.

### Example 10: Analysis of the Energy Metabolism after Administration of Azelaic Acid in Normal Mouse

Relative metabolic rates were analyzed by measuring the consumption amount of O₂ and the production amounts of CO₂ of the azelaic acid-administered group and the control group using the indirect calorimetry.

As shown in FIG. 16, in the azelaic acid-administered group, no changes of the total energy expenditure (EE) was observed compared to the control group, but the respiratory quotient (RQ) was decreased and fatty acid oxidation was increased. The decreased respiratory quotient means that fatty acid is used as a main energy source. Therefore, these above-mentioned results demonstrate that azelaic acid increased lipolysis in adipose tissue to increase the ratio of fat to be consumed as an energy source and thus the amount of the fat stored was decreased and the body fat was decreased.

A composition comprising azelaic acid, according to the present invention, has excellent effects of suppressing accumulation of lipids in adipose tissue and improving lipid metabolism in adipose tissue, such that the composition may be used as a food, a pharmaceutical composition, and a health functional food for improving the lipid metabolism in adipose tissue, such as reducing body weight and body fat (obesity alleviation).

1. A method of preventing, treating, or alleviating obesity, the method comprising administering to a subject azelaic acid, or a pharmaceutically acceptable salt thereof, or a composition containing the azelaic acid or the salt, wherein the azelaic acid or the salt acts as an agonist of olfactory receptor Olfr544 expressed in a membrane of an adipose cell.

2. The method of claim 1, wherein the azelaic acid or the salt increases triglyceride hydrolysis in adipose tissue.

3. A method of preventing, treating, or alleviating a lipid-associated metabolic disease, the method comprising administering to a subject azelaic acid, or a pharmaceutically acceptable salt thereof, or a composition containing the azelaic acid or the salt, wherein the azelaic acid or the salt acts as an agonist of olfactory receptor Olfr544 expressed in a membrane of an adipose cell.

4. The method of claim 3, wherein the metabolic disease is diabetes mellitus, hyperlipidemia, fatty liver disease, arteriosclerosis, hypertension, a cardiovascular disease, or metabolic syndrome.

5. The method of claim 3, wherein the azelaic acid increases triglyceride hydrolysis in adipose tissue.

6. A pharmaceutical or functional food composition comprising azelaic acid or a pharmaceutically acceptable salt thereof that acts as an agonist of olfactory receptor Olfr544 expressed in a membrane of an adipose cell.

7. A method of increasing intracellular cAMP, the method comprising administering to a subject azelaic acid, or a pharmaceutically acceptable salt thereof, or a composition containing the azelaic acid or the salt, wherein the azelaic acid or the salt acts as an agonist of olfactory receptor Olfr544 expressed in a membrane of an adipose cell.

8. A method of identifying whether a compound is an obesity drug candidate, the method comprising:
contacting the compound with an olfactory receptor; and
determining whether the compound acts as an agonist of the olfactory receptor.

9. The method of claim 8, further comprising determining whether the compound increases triglyceride hydrolysis in an adipose tissue.

10. The method of claim 8, wherein the olfactory receptor is Olfr544.


SEQUENCE LISTING (4 sequences)

SEQ ID NO: 1 - DNA, 20 bp, Artificial Sequence
  m olfr544_F
ggggacatctcgctgaataa

SEQ ID NO: 2 - DNA, 20 bp, Artificial Sequence
  m olfr544_R
atgaggacatggtggaggag

SEQ ID NO: 3 - DNA, 51 bp, Artificial Sequence
  m olfr544_shRNA
caccgctcactgttcgcatcttcattcgaaaatgaagatgcgaacagtgag

SEQ ID NO: 4 - DNA, 47 bp, Artificial Sequence
  scrambled shRNA
caccgtaaggctatgaagagataccgaagtatctcttcatagcctta